(19) United States
(12) Patent Application Publication
(10) Pub. No.: US 2013/0289461 A1
(43) Pub. Date: Oct. 31, 2013

(54) ORTHOSIS AND ORTHOTIC METHODS FOR APPLYING LOCALIZED FORCE TO A WEARER'S BACK

(71) Applicants: Dean E. Cropper, Ashland, OR (US); Joseph T. Zachariasen, Talent, OR (US)

(72) Inventors: Dean E. Cropper, Ashland, OR (US); Joseph T. Zachariasen, Talent, OR (US)

(21) Appl. No.: 13/843,939

(22) Filed: Mar. 15, 2013

Related U.S. Application Data
(63) Continuation-in-part of application No. 12/629,013, filed on Dec. 1, 2009, now Pat. No. 8,409,122.
(60) Provisional application No. 61/119,368, filed on Dec. 2, 2008.
(60) Provisional application No. 61/119,022, filed on Dec. 1, 2008.

(51) Int. Cl. A61F 5/02

(52) U.S. Cl. CPC A61F 5/028
USPC 602/19

(57) ABSTRACT

An back orthosis includes a lumbar panel, a support and a pair of cinching systems between corresponding sides of the lumbar panel and the support. The lumbar panel is configured to be positioned over a portion of the back of an individual's torso (e.g., over a portion of the individual's spine, etc.) and to apply localized or focused pressure to a portion of the back. The support is configured to extend over at least a portion of the front side of the torso and to be anchored by the front side. In addition, the support may include a pair of anchors that are configured to be positioned over lateral locations on the back. Each cinching system includes one or more cords and a system of rollers, or pulleys, with a set of rollers being associated with each outer edge of the support and another set of rollers being associated with each lateral edge of the lumbar panel. The rollers may be oriented non-horizontally and, along with their corresponding cord(s), may be configured maximize the amount of leverage that may be achieved with each cinching system and, thus, maximize the amount of force that the lumbar support may apply to the back.

### CROSS-REFERENCE TO RELATED APPLICATIONS

This application is a continuation-in-part of U.S. patent application Ser. No. 12/629,013, filed on Dec. 1, 2009 and titled “BACK ORTHOSIS AND ORTHOTIC METHOD” (“the '013 Application”), which claims priority under 35 U.S.C. §119(e) to U.S. Provisional Patent Application No. 61/119,368, filed on Dec. 2, 2008 and titled “BACK ORTHOSIS AND ORTHOTIC METHOD” (“the '368 Provisional Application”) and to U.S. Provisional Patent Application No. 61/119,022, filed on Dec. 1, 2008 and titled “BACK ORTHOSIS AND ORTHOTIC METHOD” (“the '022 Provisional Application”). The entire disclosures of the '022 Provisional Application, the '368 Provisional Application, and the '013 Application are, by this reference, incorporated herein.

### TECHNICAL FIELD

This disclosure relates to back braces and orthotic methods for supporting and/or relieving pain in the lower back. More particularly, this disclosure relates to back orthoses and orthotic methods that provide an adjustable level of directionally focused pressure to the lower back and high circumferential compression to the lower torso, particularly at the abdomen.

### RELATED ART

Back braces are intended to alleviate or eliminate pain and suffering caused by various disorders along a person's spine. Some spinal disorders relate specifically to the lumbar region (vertebrae L1 through L5), while other spinal disorders relate specifically to the thoracic lumbar region (vertebrae T5 through S1). Other spinal disorders stem at least in part from osteo-degenerative causes. The dynamic, integrated nature of various portions of the spine—as well as the many other variables that affect spinal disorders—make the occurrence of such disorders dependent on a large number of factors.

The alleviation of back pain sometimes includes the application of a mechanical advantage by way of a circumferential torso brace. Systems that include circumferential torso braces provide some degree of temporary relief, but they do not provide optimal pain relief. Typically, existing circumferential torso braces merely provide circumferential pressure to address back pain. For example, each of U.S. Pat. Nos. 6,213,968 B1 and 6,676,620 B2, respectively titled “Custom Fitted Orthotic Device” and “Modular Orthosis Closure System and Method” discloses a circumferential torso brace with a single pulley system that draws the brace inward toward the body in a manner that only provides basic circumferential compression, without focusing or localizing support in the lumbar area. They each have a piece that fits over the lumbar area, but none of these pieces has any connection to the workings of the brace as it is tightened other than simply being connected to the brace at the lumbar area.

U.S. Pat. No. 7,001,348 B1, titled “Double Pulley Body Brace”, discloses a circumferential torso brace with a single, one piece body panel and double pulley system, but likewise fails to provide focused or localized lumbar support or allow for lateral movement of its lumbar piece in either direction.

### SUMMARY

This disclosure is directed to a back orthosis that is easy to use and effectively reduces or eliminates back pain. In more particular embodiments, a back orthosis may be configured to provide an adjustable level of pressure to the spinal region of the lower back in a localized or focused manner. Adjustments may be made by an individual wearing the back orthosis, and while the individual is wearing the back orthosis. Such a back orthosis may be used under a variety of circumstances, including, but not limited to, addressing chronic conditions, addressing acute conditions, for pre-operative use and/or post-operative use persons, and can effectively be applied and adjusted by the wearer.

A back orthosis may include system components that broaden the brace's application such as enabling use of the back orthosis as a lumbo sacral orthosis (“LSO”), a thoracic lumbo sacral orthosis (“TLSO”) and/or an osteoarthritic (“OA”) orthosis.

The disclosure also includes an orthotic method that provides an easy and effective means to reduce or eliminate back pain.

The disclosure alternately or additionally includes an orthotic method—for chronic, acute, pre-operative, and/or post-operative persons—that enables effective application and adjustment by the wearer.

The disclosure alternately or additionally includes an orthotic method that implements system components that expand the usefulness of a brace, such as by enabling modification of the brace from an lumbo sacral orthosis (“LSO”) to a thoracic lumbo sacral orthosis (“TLSO”), an osteoarthritic (“OA”) brace, or both.

One aspect of the disclosure includes a back orthosis that includes a support, a lumbar compression piece and a pair of cinching systems. The support is configured to be positioned over at least a portion of a front side of a wearer's torso. In some embodiments, the support includes first and second, separate, opposing and mating attachment panels, which are configured to be operatively attached to one another to form a portion of a circumferential enclosure. Each attachment panel may include an outer edge with a portion that is configured to be positioned over a lateral portion of the back of the wearer's torso. The outer portion of each attachment panel is referred to herein as a “rear panel” and as a “rear anchor.”

The lumbar compression piece, which is also referred to herein as a “lumbar panel,” may form another portion of the circumferential enclosure, may be configured to apply a compressive force to a portion of an individual's back over which the lumbar compression piece resides. The lumbar compression piece is separate from the support and its position over an individual's back may be adjustable. In addition, the lumbar compression piece may be rigid. The lumbar compression piece may include opposite lateral sides.

Each cinching system includes one or more cords, as well as features of the attachment panels and the lumbar compression piece that the cord(s) interact with (e.g., eyelets or other features for receiving portions of the cords). In a specific embodiment, a first cord connects a first side of the lumbar compression piece along substantially its entire length to the first attachment panel, and a second cord connects a second side of the lumbar compression piece along substantially its entire length to the second attachment panel. The first cord may be pulled to draw the first side of the lumbar compression piece and the first attachment panel closer to one another, or loosened to space the first side of the lumbar compression piece and the first attachment panel further apart from one another. The second cord may be pulled to draw the second side of the lumbar compression piece and the second attachment panel closer to each other, or the second cord may be loosened to enable the second side of the lumbar compression piece and the second attachment panel to be spaced further apart from one another. As each cord is pulled, the brace circumferentially tightens and the cord provides a mechanical advantage that concentrates an inward compression of the lumbar compression piece, or panel, directly and especially upon a spinal region of the wearer's back. The extent of the mechanical advantage depends at least partially upon a number of apertures through which each cord passes.

In another aspect, pulling the first cord or the second cord causes relatively greater compression directly and especially upon a spinal region of a wearer's back than upon a region adjacent to the spinal region of a wearer's back.

In yet another aspect, the first set, the second set, or both first and second sets of apertures are formed by raised eyelets that are substantially parallel to the wearer's back surface when the lumbar compression piece is worn, and provide additional anchoring leverage actively to force the lumbar compression piece specifically at the spinal region and so inwardly compress the spinal region of the wearer's back while keeping the first cord and/or the second cord spaced away from the wearer's back at a region adjacent to the spinal region of the wearer's back.

In still another aspect, the first and second, separate, opposing and mating front attachment panels have anterior portions that have greater rigidity than lateral sides of the orthosis, which provides an opposing vice-like frontal force diametrically opposed to the concentrated inward compression of the separate, laterally adjustable, lumbar compression piece directly and especially upon a spinal region of a wearer's back.

In yet another aspect, the separate, laterally adjustable, lumbar compression piece has a raised, beveled inner surface that operatively abuts against the wearer's spinal region.

In still another aspect, the lumbar compression piece has a flat, curved, regular, irregular, or form-fitting inner compression surface and a generally flat, curved, regular, irregular, or form-fitting outer surface.

In yet another aspect, the lumbar compression piece has at least four slots on each of the first and second lateral sides by which the respective first and second cords engage the lumbar compression piece so causing the lumbar compression piece to contour to a shape of the wearer's back.

In yet another aspect, the back orthosis further comprises at least one tensioning handle with respect to which the first cord passes and with respect to which a portion of the first cord changes its relative position in accordance with variations in upper and lower first cord material lengths, relative to one another, each of which runs between the at least one tensioning handle and the first set of apertures.

In still another aspect, the first cord changes its position with respect to the tensioning handle at least in accordance with adjustable vertical and circumferential stationary tensioning handle attachment positions on the first or second, separate, opposing and mating front attachment panels, or first and second corresponding orthosis side portions.

In yet another aspect, the back orthosis further comprises a pad positioned between the wearer's back and the lumbar compression panel wherein foam within the pad flows.

In still another aspect, the lumbar compression piece comprises a horizontally and vertically centered reinforced center grid bordered by upper and lower hollowed out, beveled bowls, the bottoms of which push against the wearer's spinal region.

In yet another aspect, the first lateral side of the separate rigid lumbar compression piece is immediately adjacent to at least one vertically centered aperture, through which the first cord passes through a final aperture on the separate lumbar compression piece just before extending to a manually operated tensioning handle.

In still another aspect, the at least one vertically centered aperture is a pair of side-by-side apertures, through each of which one of two opposing lengths of the first cord passes.

In yet another aspect, the separate, laterally adjustable, lumbar compression piece includes a central vertical axis about which independent horizontally disposed extensions project.

In still another aspect, the independent, horizontally disposed extensions each correspond to at least one aperture of the first and second sets of apertures.

In yet another aspect, the independent, horizontally disposed extensions operatively flex in accordance with varying contours of lumbar and thoracic spinal regions that vary from individual-to-individual, with a finger-like horizontal and transverse motion when engaged in tension by the first and second cords.

Another aspect of the disclosure includes a back orthosis comprising a lumbar compression piece configured for self-adjustable positioning only at the rear of a wearer; twin opposing rigid panels, which are also referred to as “rear panels” and as “rear anchors,” for immobile placement upon opposing lateral sides of a spinal region of the wearer; a front abdominal piece, configured for immobile positioning upon an anterior side of the wearer; and at least two independent cinching systems, at least two of which are on opposing lateral sides of the lumbar compression piece, each cinching system providing a mechanical advantage for circumferential tightening of the orthosis and applying a relatively greater force than a circumferential force specifically to four points of the wearer's torso; wherein the lumbar compression piece and rear panels direct pressure radial to the wearer's spine; and wherein three of the four points are immobile while the forth point is laterally movable about a posterior lumbar side of the wearer, and the lumbar compression piece addresses the forth point of the wearer's torso.

In another aspect, the disclosure includes a back orthosis comprising first and second attachment panels and a lumbar compression piece that includes: opposing lateral sides, one such side connected along substantially its entire vertical length to the first attachment panel by a cord, and the other such side connected along substantially its entire vertical length to the second attachment panel by another cord or by an elastic piece; a first set of apertures through which the cord passes and is operatively pulled or allowed to retract, respectively either to draw the one such side laterally closer to the first attachment panel or to distance the one such side laterally further from the first attachment panel; wherein pulling of the cord causes the brace to circumferentially tighten and to concentrate compression of the lumbar compression piece directly and especially upon a spinal region of a wearer's back with the aid of a mechanical advantage, which may be dependent upon a number of apertures through which the cord or the another cord passes.

Another aspect of the disclosure includes an orthotic method comprising utilizing a back orthosis, comprising first and second attachment panels, which are operatively attached to one another to form a circumferential enclosure about a wearer; and a lumbar compression piece including: opposing first and second lateral sides, the first side connected along substantially its entire vertical length to the first attachment panel by a first cord, and the second side connected along substantially its entire vertical length to the second attachment panel by a second cord; a first set of apertures through which the first cord passes and is operatively pulled or allowed to loosen, respectively either: to draw the first side laterally closer to the first attachment panel, and the first attachment panel posteriorly closer to the first side, or to distance the first side laterally further from the first attachment panel, and the first attachment panel further from the first side; and a second set of apertures through which the second cord passes and is operatively pulled or allowed to loosen, respectively either: to draw the second side laterally closer to the second attachment panel, and the second attachment panel posteriorly closer to the second side, or to distance the second side laterally further from the second attachment panel, and the second attachment panel further from the second side; wherein pulling of the first cord and the second cord causes the brace both to circumferentially tighten and to concentrate inward compression of the lumbar compression piece directly and especially upon a spinal region of the wearer's back with the aid of a mechanical advantage, which may be dependent upon a number of apertures through which the first cord or the second cord passes.

### BRIEF DESCRIPTION OF THE DRAWINGS

In the accompanying drawings, which form a part of the specification and are to be read in conjunction therewith, and in which like reference numerals are used to indicate like parts in the various views:

FIG. 1 is a plan view of an inner side of an embodiment of a back orthosis 1 with a partial cutaway view of a perforated rigid front panel;

FIG. 2 is a plan view of an outer side of the back orthosis 1 of FIG. 1;

FIG. 3 is a top partial cross-sectional view of a person's torso, including several relevant anatomical features in a plane perpendicular to a vertical spinal axis, subjected to enhanced and uniquely applied posterior-to-anterior forces by the lumbar panel 3, rear panels 33 and 35, and front attachment pieces 19 and 21 of the back orthosis 1 of FIG. 1;

FIGS. 3a-d schematically depict how the angle at which the rear panels 33 and 35 are oriented affects the force applied by the lumbar panel 3;

FIG. 4 is a side partial cross-sectional view of a person's torso, showing a spine in a plane sagittal to a vertical spinal axis that is subjected to enhanced and opposing inward forces by the opposing lumbar panel 3, rear panels 33 and 35, and front attachment panels 19 and 21 of the back orthosis 1 of FIG. 1;

FIG. 5 is a partial lower perspective view of one of the cinching systems of the back orthosis 1 of FIG. 1;

FIG. 6 is a perspective view of another embodiment of a cinching system 8′ that may be included in a back orthosis, such as that depicted by FIG. 1, which cinching system 8′ includes vertically oriented rollers 89′ that guide the movement of cords 9′ and 10′ that extend between a lumbar panel 3′ and corresponding rear panels 33′ and 35′ adjacent to opposite sides of the lumbar panel 3′;

FIG. 6a is a perspective assembly view of the embodiment of cinching system 8′ show in FIG. 6;

FIG. 6b is a cutaway view showing the arrangement of the rollers 89′ and cords 9′ and 10′ relative to the adjacent sides of the lumbar panel 3′ and its corresponding side panels 33′ and 35′;

FIG. 7a is a rear view of a torso of a person wearing the back orthosis 1 of FIG. 1, wherein the lumbar panel 3 is adjusted to apply force at the left side of the person's spinal region;

FIG. 7b is a rear view of a torso of a person wearing the back orthosis 1 of FIG. 1, wherein the lumbar panel 3 is adjusted to apply force at the center of the person's spinal region;

FIG. 7c is a rear view of a torso of a person wearing the back orthosis 1 of FIG. 1, wherein the lumbar panel 3 is adjusted to apply force at the right side of the person's spinal region;

FIGS. 8a-c are respective side, top partial cut-away, and bottom views of a tensioning handle of the back orthosis 1 of FIG. 1;

FIGS. 9a and 9b are respective attachment and application sides of a foam lumbar pad;

FIG. 10 is a partial side assembly view of the back orthosis 1 of FIG. 1 and the foam lumbar pad of FIGS. 9a and 9b;

FIG. 10a is plan view of an attachment side of a thoracic lumbo sacral attachment panel;

FIG. 10a-1 is a cut away side view of the clip tab 127 shown in FIG. 10a;

FIG. 10b is a plan view of an attachment side of another embodiment of a thoracic lumbo sacral attachment panel;

FIG. 11 is a side view of the lumbar panel 3 of FIG. 1 and the thoracic lumbo sacral attachment panel of FIG. 10a, as curved along their lengths when in use;

FIG. 12a is a plan view of the posterior side of one embodiment of a flexible multi-positional lumbar panel;

FIG. 12b is a perspective view of the lumbar panel of FIG. 12a and a foam pad, as incorporated into an alternate embodiment of the back orthosis 1 of FIG. 1.

### DETAILED DESCRIPTION

As illustrated in the accompanying drawings and discussed in detail below, one aspect of this disclosure includes a back orthosis that, when used, provides high circumferential compression to the lower torso with a high level of mechanical advantage. The back orthosis of this aspect nevertheless also applies a continued, direct, adjustable amount of active support especially to the lower back, which can be finely tuned by lateral adjustment specifically to a particular area of pain.

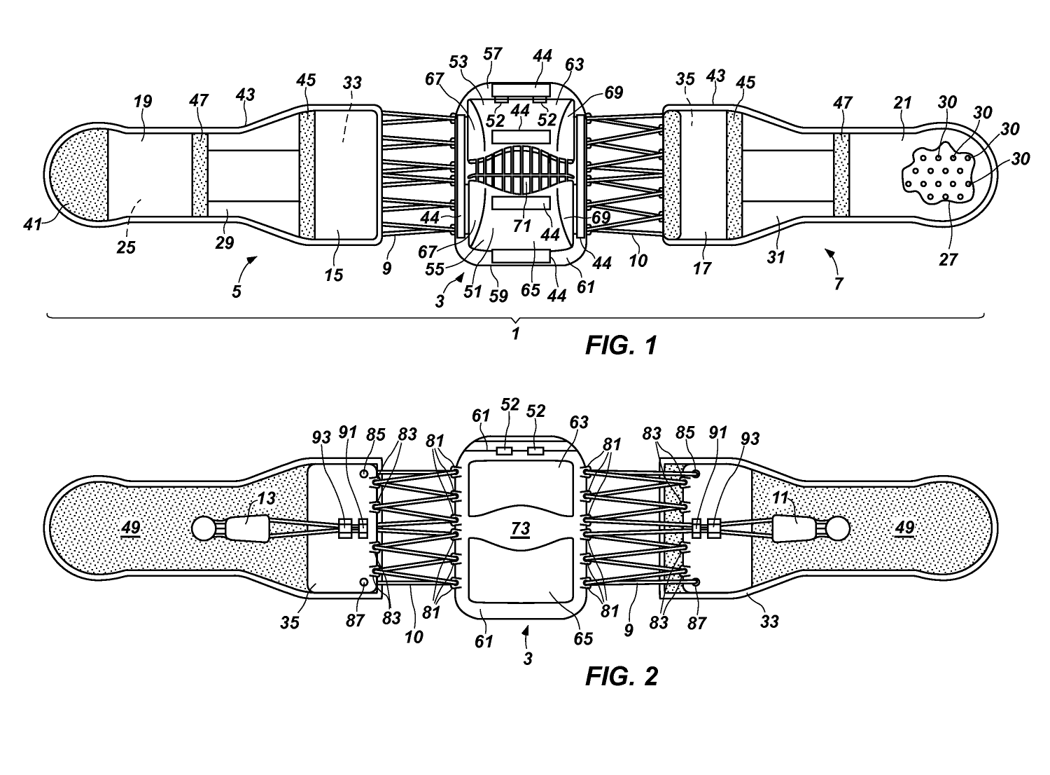

In one embodiment, the back orthosis 1 is a lumbo sacral orthosis (“LSO”). Referring to FIGS. 1 and 2, back orthosis 1 generally includes rigid separate lumbar panel 3, which is attached to each of right side attachment member 5 and left side attachment member 7 by respective separate right and left cords 9 and 10 to form a circumferentially attachable orthosis configured to be positioned about a wearer's waist and torso. Right and left tensioning handles 11 and 13 can be used by any wearer to tighten and loosen cords 9 and 10, which run between lumbar panel 3 and respective right 5 and left 7 side attachment members. After extensive research, it has been discovered moreover that back orthosis 1 thereby not only provides an overall system that increases compression about the torso, it provides a unique and surprisingly effective multiple-independent-point, symmetric anchoring system that specifically and inwardly compresses the wearer's spine and abdomen in a vice-like, diametrically-opposed, planar fashion, which unloads the spine to alleviate pain while allowing side-to-side movement of lumbar panel 3.

Lumbar panel 3 fits directly over the lumbar spinal region of a wearer's back, including the spine and the soft tissue surrounding it. When applied as described below, it acts as a posterior wall to the lumbar spine in such a way that when the below-described cinching system is drawn, lumbar panel 3 creates an inward force against the spine and soft tissue surrounding the spine. This helps to decompress the spine with the help of a continuous opposing force from at least one attachment panel 19 and 21, while lumbar panel 3 is free to laterally move side-to-side and about a wearer's spine in accordance with a particular spinal compression and pain relief for a given situation at a particular time.

Lumbar panel 3 is specifically shaped and configured to maximize its effectiveness to accomplish these and other ends. Inner side 51 of lumbar panel 3 is generally shaped as an inverted rectangular dish comprising upper and lower symmetrically oriented bowls 53 and 55. Upper and lower side walls 63 and 65 run towards the panel's center approximately from perimeter (approximately 1-inch wide) to lip 61 (which is at least approximately parallel to the wearer's back when worn and is rounded at its corners). Specifically, from respective horizontal sides 57 and 59, walls 63 and 65 form relatively obtuse angles (with respect to immediately adjacent portions of lip 61) when compared to the angles formed between right and left lateral walls 67 and 69. Thus, the walls 63 and 65 are gently sloped relative to the walls 67 and 69.

Dividing and reinforcing bowls 53 and 55 is a vertically and horizontally centered reinforcement section 71, specifically comprised of a hollow raised lattice work (defining approximately 48½ inch segments of a various assortment of full or truncated squares) that fills the space that occupies the dish volume otherwise defined by bowls 53 and 55. When compressed by application of back orthosis 1, lumbar panel 3 flexes along the vertical curvature of a wearer's spine, at which point this reinforcement section 71 provides added support and strength to lumbar panel 3. Section 71 also provides support and strength when lumbar panel 3 does not vertically flex. Posterior plate 73 adds further support.

The resulting lumbar panel 3, which is generally vertically elongated, provides dual, centrally and inwardly thrust projections each having vertically and laterally beveled interior sides, both (1) generally to decompress a spine in a direction normal and perpendicular to the plane generally created by a wearer's back, but (2) also about the axis of the spine from between about 0° to about 45° (or from about 0° to about 5°, 10°, 15° or 20°) angles with respect to a position parallel to such plane.

Lumbar panel 3, especially when used with the cinching systems described below, projects inwardly and thereby applies particularly enhanced (as relative to the circumferential intra-cavity pressure otherwise exerted about a wearer) inwardly focused, concentrated force towards a wearer's spinal region. As described below, this relatively greater force against the spinal region is continually radial to the spinal region even as lumbar panel 3 is positioned at laterally varied locations from side-to-side across a wearer's back, because of the specially adapted beveled walls 67 and 69 of bowls 53 and 55, and the various possible, separately adjustable, angular positions of lumbar panel 3 with respect to the plane parallel to a wearer's back.

This configuration is especially and surprisingly effective when lumbar panel 3 is integrated into the workings of two or more laterally opposing independent and immobile cinching systems, as described below.

In one embodiment, two or more independent cinching systems may be used with lumbar panel 3. The cinching systems may be configured to cinch laterally, vertically and/or diagonally.

Lumbar panel 3 is also configured to accept intermediate self-attaching comfort pads, e.g., warm or cold gel pads, used for heat or cold therapy—as discussed further below. For example, hook or loop-friendly fabric strips 44 allow such removable attachment.

Lumbar panel 3 is optionally also configured with slots 52 that receive TLSO or OA orthosis attachments for secured removable attachment and therapy, also discussed below, though another embodiment has no such slots.

Each right 5 and left 7 side attachment member comprises a back segment 15 (or 17), an attachment panel 19 (or 21), and a lateral segment 29 (or 31). Each back segment 15 and 17 contains and confines a rear panel 33 (or 35), which may be rigid (e.g., it may comprise a plastic, etc.) and may be sewn into its corresponding back segment 15 (or 17) to keep the rear panel 33 (or 35) from moving laterally, vertically, diagonally, or any combination thereof. Rear panels 33 and 35 are specifically bound by stitching in perimeter borders 43 and partitioning borders 45. This placement, which is immovable with respect to the rest of a respective attachment member 5 or 7, maintains proper anchor positioning of its corresponding back segment 15 and 17 with respect to a wearer's body. Attachment panels 19 and 21 include front panels 25 and 27, which may comprise rigid elements with perforations 30, and likewise contain and confine front panels 25 and 27 for the same reasons at stitched perimeter borders 43 and partitioning border 47. As shown in FIG. 1, front panel 27 has its outside nylon or polyester foam fabric 101 partially cut away. Exposed front panels 25 (not shown) and 27 have regular ¼ inch circular perforations, which provide structural integrity.

Each rear panel 33, 35 provides an independent rear anchoring point at a location lateral to a wearer's spinal region. They are not configured to be positioned directly adjacent to the wearer's spinal region. Rather, they are configured to be positioned directly over either right or left lateral muscle groups, i.e., over two muscle groups in the low back, the transversospinalis and intertranversarri portions of the wearer's back. This positioning provides additional inward pressure in parallel opposition to the force applied by front panels 25 and 27. This positioning also prevents axial or rotational displacement of the back orthosis 1 about the wearer's body when worn. Combined with lumbar panel 3, they provide two symmetric points of a three point stabilizing system, as discussed further below. Rear panels 33 and 35 further provide a guide and anchor position for the cinching systems that include cords 9 and 10.

Lateral segments 29 and 31, which may comprise flexible elements, are configured to circumferentially wrap around a wearer's torso, and so provide a flexible yet durable connection between the back segments 15 and 17, and their corresponding attachment panels 19 and 21. Lateral segments 29 and 31, and the exteriors of back segments 15 and 17 and attachment panels 19 and 21, may be made from an external durable woven nylon formed as loop material laminated onto a polyester foam laminated onto a brushed nylon fabric. This protects the internal components, but any durable and strong relatively inelastic fabric may be used.

Monolithic, one-piece lumbar panel 3 (including eyelets 81) may be made entirely from injection molded nylon having a relatively firm hardness, but any rigid plastic material or similarly functioning material may be used, such as polyethylene, polyvinylchloride, or any other polymer or co-polymer resin. For example, treated natural wood, any foam injection resins (such as comprised of the polymers stated above), hard rubber, composites, metals, or many other materials may be used. In one embodiment, lumbar panel 3 is made from duPont ZYTEL® 66 nylon (as described in U.S. Pat. No. 6,756,429 herein incorporated by reference in its entirety) having a Shore D hardness of about 85, or from about 80 to about 90. Any material suitable for performing any or all of the functions stated herein may be used, however.

The lumbar panel 3 may be about ½ inch thick but, as described above, may vary in thickness across its unique shape, including at locations that include structural reinforcement. In its panel form, lumbar panel 3 may vary in general thickness from about % inch to about 2 or more inches, but of course carbon composite or high strength metal panels would allow for thinner or thicker panels to provide similar function.

Monolithic, one-piece rear panels 33 and 35 (including eyelets 83) may be made from injection molded nylon having a relatively firm hardness, but any rigid plastic material or similarly functioning material may be used, such as polyethylene, polyvinylchloride, or any other polymer or co-polymer resin. For example, treated natural wood, any foam injection resins (such as comprised of the polymers stated above), hard rubber, composites, metals, or many other materials may be used. In one embodiment, rear panels 33 and 35 are made from duPont ZYTEL® 66 nylon (as described in U.S. Pat. No. 6,756,429, the disclosure of which is, by this reference, hereby incorporated herein in its entirety) having a Shore D hardness of about 85, or from about 80 to about 90. Any material suitable for performing any or all of the functions stated herein may be used, however.

Front panels 25 and 27 may be made from ¼ inch injection molded polyethylene, but may be made from anywhere between about % and about ¾ inch. As hardness varies, so may the relative thickness of each front panel 25, 27, as long as it achieves some or all of the functions disclosed herein.

Nearly all of the components of back orthosis 1 may be symmetrical about a plane running through the vertical central axis of lumbar panel 3, except for an additional piece of hook-friendly fabric 41 used to attach right attachment member 5 over and onto left attachment member 7.

In an alternate embodiment, left attachment member 5 fits over right attachment member 7, however.

Outer sides 49 are covered in hook-friendly fabric, which mates with both loop-friendly fabric piece 41 on attachment panel 19 and loop-friendly material on the inner surfaces of right and left tensioning handles 11 and 13. Hook and loop fabric on any of the mating components may be reversed, or other suitable reversibly self-adjustable and attachable fabrics or fasteners may be used.

In another embodiment, attachment members 5 and 7 are identically equipped with various other types of attachment materials or devices suited for the adjustable attachment called for herein wherein attachment members 5 and 7 provide a rotationally and vertically immobile fit.

In any case, the various components of attachment members 5 and 7—in particular rear panels 33 and 35, and mating attachment panels 19 and 21—when attached as part of back orthosis 1, may be rotationally and vertically immobile with respect to the wearer's torso by virtue of the panel and fabric specifications noted above, and the relative placement and structural configuration described herein below.

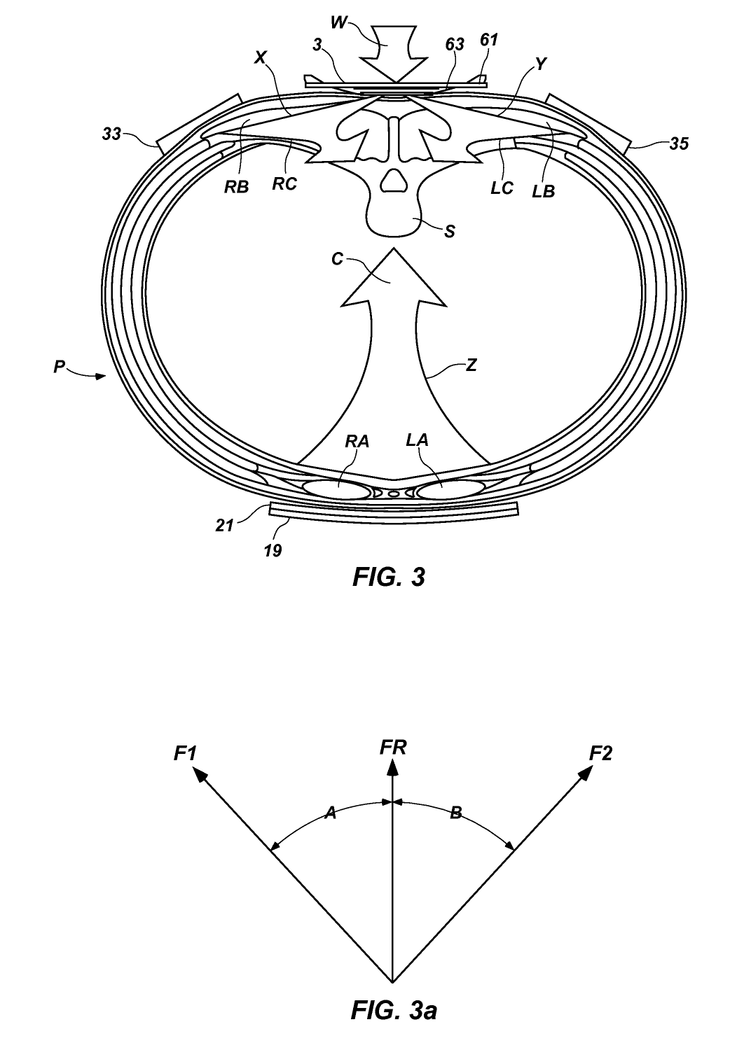

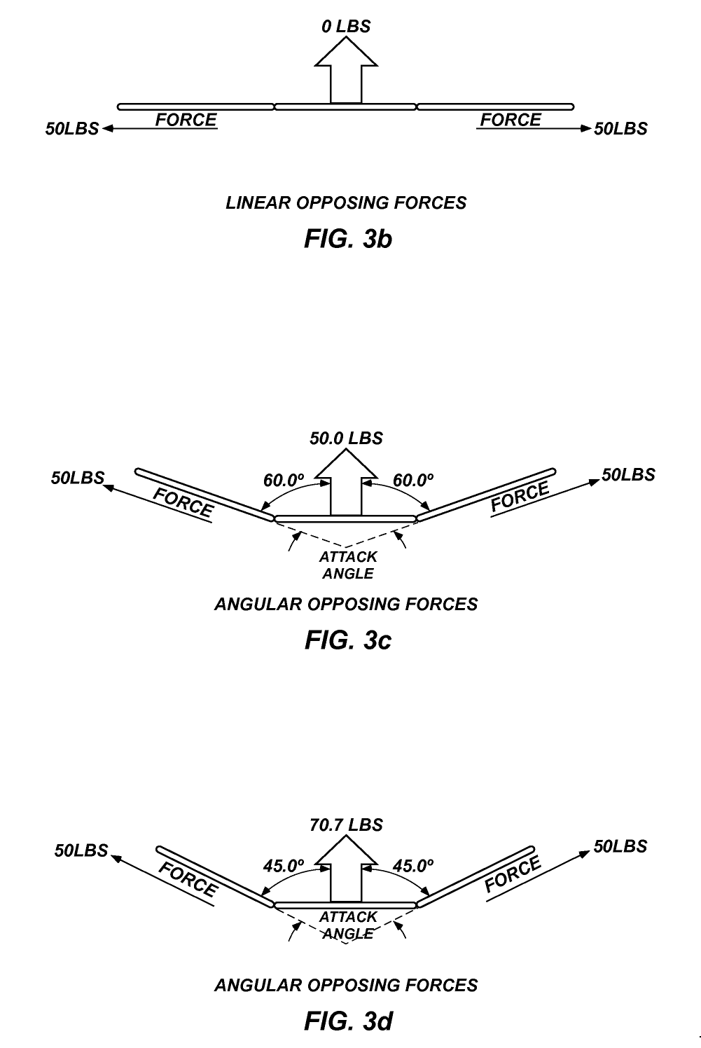

Back orthosis 1 provides at least three fixation points in a three-point stabilizing system. Referring to FIG. 3, when rear panels 33 and 35 are rotationally and vertically immobilized and front panels 25 and 27 (FIG. 1) are rotationally and vertically immobilized, each of these panels provides one point of three immobile anchoring points in a four point anchoring system. These three points alone provide diametrically opposing, vice-like parallel force particularly upon the abdomen and spine S to decompress the spine S. The lumbar panel 3 provides direct active force upon spine S, however. The rear panel 33 on the right provides direct force upon right transversospinalis, intertranversarri, interspinales, intertransversarii mediales, multifidi, and lumbar erector spinae areas RC, with relatively less pressure applied to an area just adjacent to the right side of the spine RB. The rear panel 35 on the left provides direct active force upon left transversospinalis, intertranversarri, Interspinales, intertransversarii mediales, multifidi, lumbar erector spinae areas LC, with relatively less pressure applied to area just adjacent to the left side of the spine LB.

Thus, lumbar panel 3 alone, or when coupled with the right rear panel 33 and the left rear panel 35, provides posterior force parallel to a wearer's back. While lumbar panel 3 may be adjusted from side-to-side, rotationally and vertically immobilized rigid front panels 25 and 27 (FIG. 1), and immobilized rear panels 33 and 35 stay unmoved. This is illustrated by the unchanging inward but also directly opposing directional arrows W, X, Y, and Z, which are consistently, continually and continuously, approximately or precisely, radially directed or focused not strictly at intra-cavity center C of cavity P, but rather toward spinal region S. This focusing is achieved in part by applying force at specific points along the circumference of cavity P. Notably, such force is not primarily directed at muscles RB and RB, but rather bone structures RC, LC and S, and diametrically opposed abdominal muscles BA and LA, and is achieved by the arrangement of four panels at four anchoring points.

With such an arrangement, the amount of force applied by the lumbar panel 3 is defined at least partially by the angle at which the rear panels 33 and 35 are oriented relative to one another, which is referred to as an “attack angle.” As illustrated by FIG. 3a, when force is applied from two separate locations, or from force vectors F1 and F2 (e.g., from the rear panels 33 and 35 to the sides of the lumbar panel 3, etc.), the combined force, or resultant force vector FR (e.g., the amount of force applied by the lumbar panel 3, etc.) is defined by the values of force vectors F1 and F2, and by the angles between force vector F1 and the resultant force vector FR and between the resultant force vector FR and force vector F2, as follows:

FR=[F1²+F2²−(2×F1×F2×cos(180°−(A+B))×½, (1)

where F1 is the amount of force applied along force vector F1, F2 is the amount of force applied along force vector F2, A is the measure of the angle between force vector F1 and the resultant force vector FR, and B is the measure of the angle between the resultant force vector FR and force vector F2. As illustrated by FIG. 3b, when force is applied from opposite directions, there is no force along the desired resultant force vector FR. As the measure of the angle A, B between each force vector F1, F2 (e.g., each rear panel 33, 35, etc.) and the desired resultant force vector FR (e.g., the lumbar panel 3, etc.) decreases, however, the amount of force applied along the resultant force vector FR (e.g., by the lumbar panel 3, etc.) increases, as shown by FIGS. 3c and 3d.

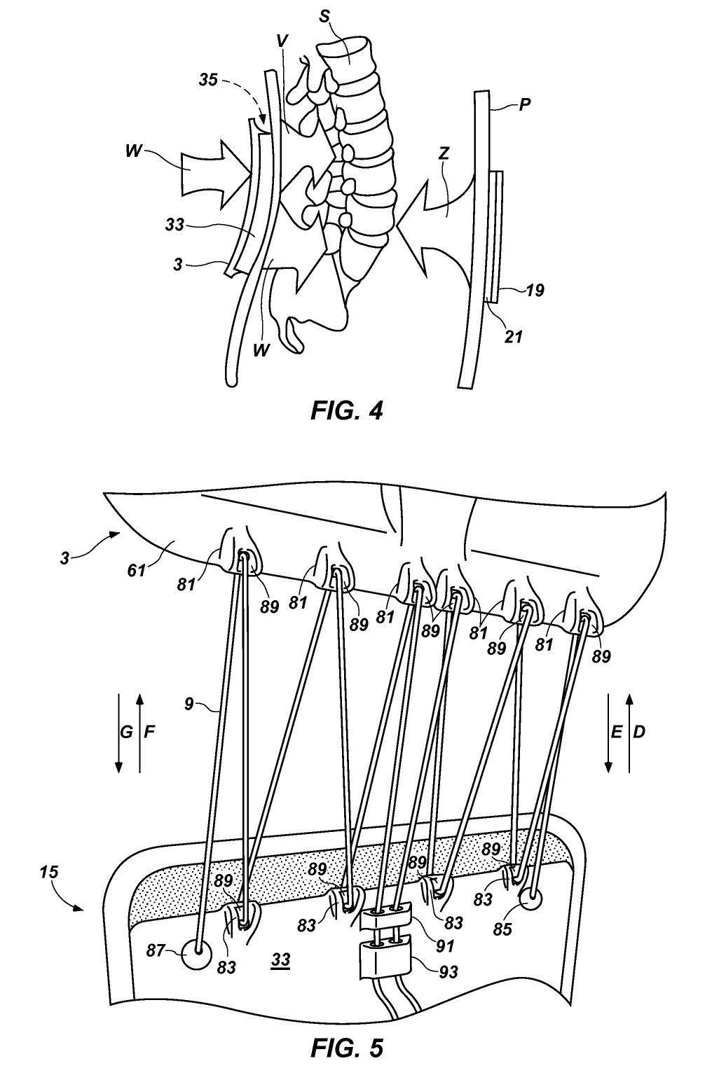

Referring to FIG. 4, the three immobile anchoring points upon which the front panels 25 and 27 (FIG. 1) and the rear panels 33 and 35 address a wearer, and a movable anchoring point upon which (when attached) the lumbar panel 3 addresses a wearer also provide various degrees of compression directed at spinal region S, that vary along the sagittal plane, e.g., a plane running vertical to divide right and left sides of cavity P. Cords 9 and 10, as shown in FIGS. 1 and 2 and described in further detail below, may be cinched with varying degrees of tightness from the upper 53 to lower 51 sections (FIG. 1) of the lumbar panel 3. As more tension is introduced into one or both of the cords 9 and 10, the rear panels 33 and 35 may remain at the same locations or substantially the same locations on the body of the wearer, while the attack angle between the rear panels 33 and 35 increases, increasing the amount of force applied by the lumbar panel 3 (see equation (1); FIGS. 3a-d). Conversely, as tension in one or both cords 9 and 10 decreases, so does the attack angle at which the rear panels 33 and 35 are oriented, which decreases the amount of force applied by the lumbar panel 3. As therapeutically needed, variation in the amount of force applied by the lumbar panel 3 along the sagittal plane is illustrated by directional arrows V and W, which, like arrows W, X, Y and Z, concentrate, or focus, a greater amount of relative pressure directly upon and inwardly toward the spine S, and not simply toward the cavity center C. The application of force by the lumbar panel 3 toward the spine S may be accompanied by providing a diametrically opposed, posterior and anterior, repeatedly and easily self-adjustable, parallel vice-like force to discrete, spaced apart locations of the wearer's torso.

Referring to FIG. 5, though three points of the four-point anchoring system are immobilized once back orthosis 1 (FIG. 1) is attached, lumbar panel 3 is employed as part of an self-adjustable, dual, independent cinching system that provides additional diametrically opposing parallel-vice-like force particularly upon the abdomen and spine to decompress the spine. Even after temporarily immobile attachment of back orthosis 1, nearly any conscious wearer—almost regardless of strength or condition—can adjust the side-to-side positioning and relative upper and lower force at which lumbar panel 3 is applied. Lumbar panel 3 is connected with two independent cord systems that can move side-to-side because they are both independent and relatively raised from the back. This allows application of directed pressure on top of a particular acute or chronic pain location just lateral to the spine, for example in the case of a muscle spasm. At any lateral position, lumbar panel 3 consistently and continually applies force that is radial to the wearer's spine, and not simply the center of the torso cavity.

A set of six raised eyelets 81 on each side of lumbar panel 3 and four raised eyelets 83 on each of rear panels 33 and 35 (only rear panel 33 is shown in FIG. 5), further separate cords 9 (or 10) from back orthosis 1 (FIG. 1), and thus the wearer, thereby to provide added leverage to employ each of the dual independent, cinching systems. Each such system includes lumbar panel 3, cords 9 or 10, respective rear panels 33 or 35, and tensioning handles 11 or 13 (FIG. 2). One end of cord 9 may be anchored to rear panel 33 (e.g., it may be knotted and fed through attachment hole 85 to secure it to rear panel 33, etc.). Cord 9 runs between the upper half of alternating eyelets 81 and 83, through cord guides 91 and 93 and through tensioning handle 11. Then cord 9 runs back through cord guides 93 and 91 and through the lower half of alternating eyelets 81 and 83—each time passing through acutely angled guide grooves 89.

Thus, raised eyelets 81 and the bowl portions of lumbar panel 3 provide several unique advantages. Lumbar panel 3 is compressed against a spinal region as cords 9 and 10 are being tensioned and the pressure is coming from rear panels 33 and 35. At the same time, rear panels 33 and 35 are being pulled up away from the back as cords 9 and 10 are being tensioned, due to the raised portions of lumbar panel 3. The two offset one another so the pressure under the rear panels 33, 35 and lumbar panel 3 can be effectively the same. The effects on the underlying soft tissue can remain fairly constant under both areas.

In one embodiment eyelets 81 are replaced by simple holes having rounded and slick surfaces, or any other type of apertures through which cords 9 and 10 may pass to achieve a mechanical advantage.

In one embodiment, grooves 89 are angled from about 40° to about 70°, and in another embodiment grooves 89 are angled from about 45° to about 65°.

As such, lumbar panel 3 and rear panel 33 move and separately in accordance with the relative, and counteracting tightening and loosening of cords 9 and 10. This tightening, which may vary from the upper to the lower communicating portions of lumbar panel 3 and rear panel 33, will cause separately adjustable distancing along the entire vertical side of lumbar panel 3, as indicated by various directional movement arrows D, E, F, and G.

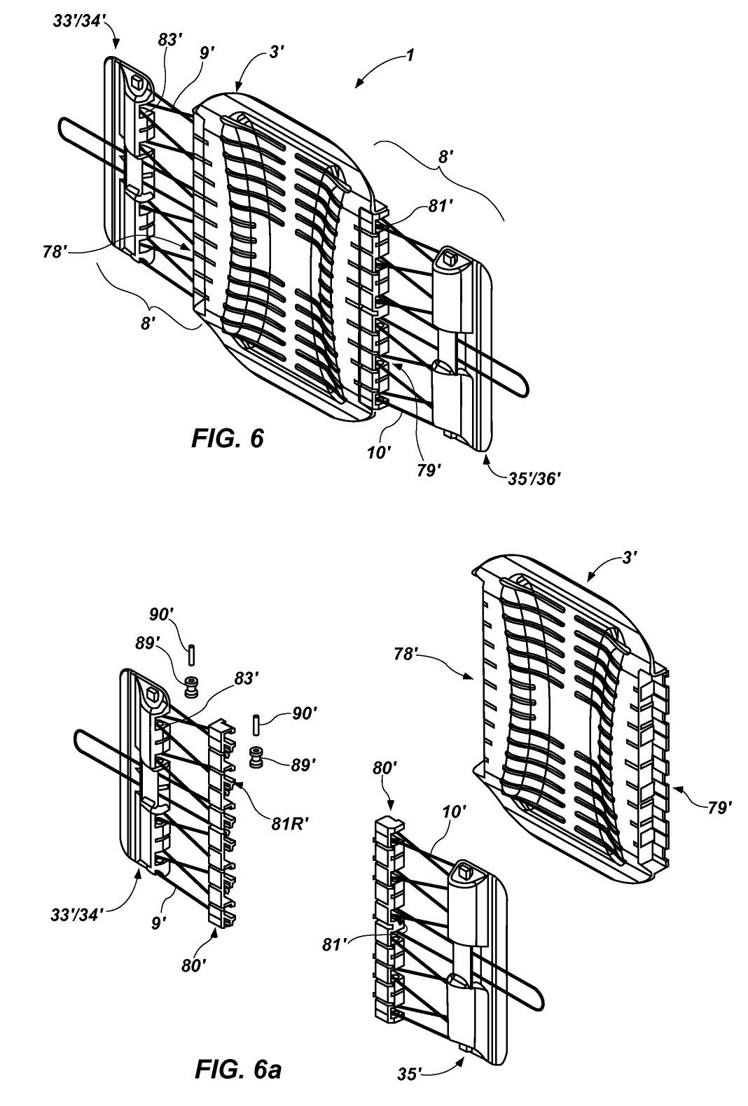

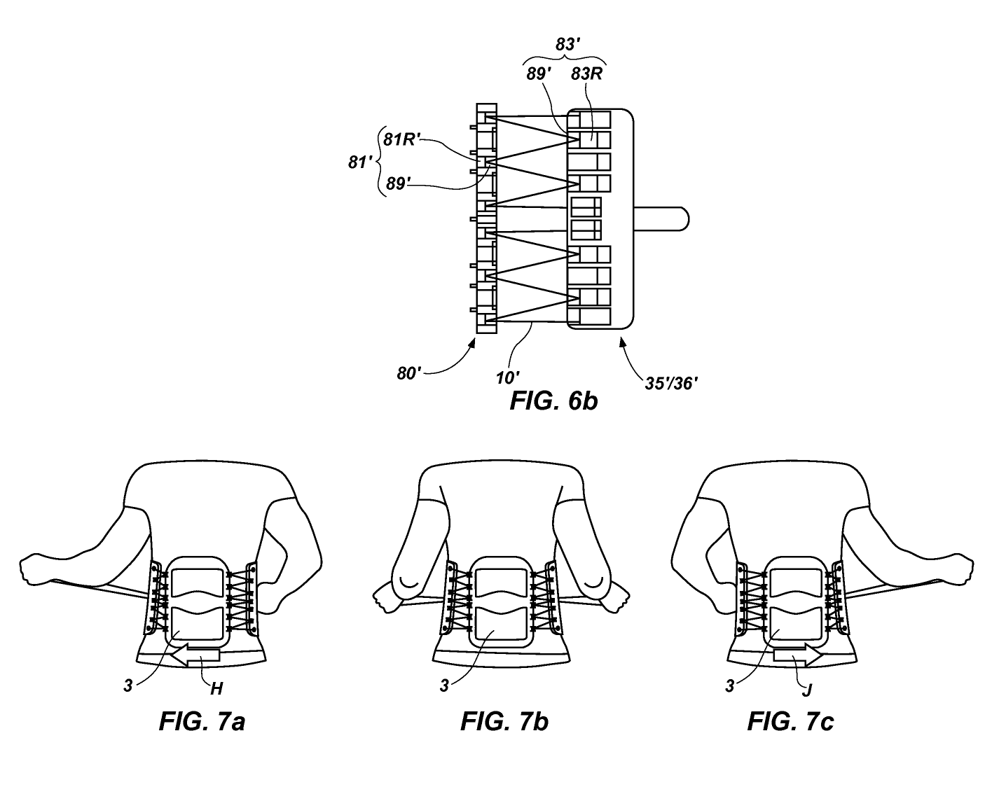

FIG. 6 is a perspective view of another embodiment of a cinching system 8′ that may be included in a back orthosis 1, such as that depicted by FIG. 1. The cinching system 8′ includes eyelets 81′ of the lumbar panel 3′, eyelets 83′ of each rear panel 33′, 35′, a cord 9′ laced through the eyelets 83′ of rear panel 33′ and the eyelets 81′ on side 78′ of the lumbar panel 3′ located adjacent to rear panel 33′, and a cord 10′ laced through the eyelets 83′ of rear panel 35′ and their corresponding eyelets 81′ on the side 79′ of the lumbar panel 3′ located adjacent to rear panel 35′. Each cord 9′, 10′ may be adjusted in a manner that positions its corresponding rear panel 33′, 35′ and the lumbar panel 3′ in a desired orientation relative to one another and at a desired distance from one another. Thus, the cords 9′ and 10′ and the eyelets 81′ and 83′ through which they extend may be adjusted to provide a desired amount of tension, and to localize or focus force and pressure in a desired direction onto a desired portion of a wearer's body.

As illustrated by FIGS. 6a and 6b, the positions (e.g., elevations, etc.) of eyelets 81′ on each side 78′, 79′ of the lumbar panel 3′ may be offset relative to, and alternate with the positions of, their corresponding sets of eyelets 83′ on the rear panel 33′, 35′ adjacent to that side 78′, 79′. Of course other arrangements of adjacent sets of eyelets 81′ and eyelets 83′ are also within the scope of the disclosed subject matter.

Each eyelet 81′, 83′ may include a receptacle 81R′, 83R′ configured to receive a roller 89′ and a pin 90′. The pin 90′ serves as an axis about which the roller 89′ rotates. In the embodiment shown in FIGS. 6a and 6b, the eyelets 81′ on each side 78′, 79′ of the lumbar panel 3′ may be defined by a roller gate 80′, which is in turn located at or adjacent to a side 78′, 79′ of the lumbar panel 3′, or to be assembled and secured at or adjacent to a side 78′, 79′ of the lumbar panel 3′. Each receptacle 81R′ may be configured in such a way as to receive and retain (e.g., by snap fit, etc.) the ends of a pin 90′ that extends through the axis of rotation of a roller 89′, and in a manner that enables the roller 89′ to rotate freely about its corresponding pin 90′. Separate roller gates 34′ and 36′, which comprise parts of medial portions of the rear panels 33′ and 35′, or which may be configured for assembly with the rear panels 33′ and 35′, respectively, include eyelets 83′ with receptacles 83R′ that may be configured in the same manner as or in a similar manner to the receptacles 81R′ of the eyelets 81′ of the lumbar panel 3′.

Cords 9′ and 10′ are laced through the eyelets 81′ and 83′. In the specific, but non-limiting embodiment depicted by FIGS. 6-6b, the ends of each cord 9′, 10′ are secured, at anchor points 85′ and 87′, respectively, to upper and lower positions along the height of the roller gate 34′, 36′ of the rear panel 33,′ 35′ that corresponds to that cord 9′, 10′. As shown, an upper portion of each cord 9′, 10′ may be laced in a zig-zag fashion through the eyelets 81′ of an upper portion of its corresponding side 78′, 79′ of the lumbar panel 3′ and through the eyelets 83′ of an upper portion of its corresponding roller gate 34′, 36 of a rear panel 33′, 35′. A central portion of each cord 9′ and 10′ may be laterally accessible from a central portion along the height of each rear panel 33′ and 35′. A lower portion of each cord 9′, 10′ extends between lower portions of its corresponding side 78′, 79′ of the lumbar panel 3′ and the roller gate 34′, 36′ of its corresponding rear panel 33′, 35′, and through the respective eyelets 81′ and 83′ of the lower portions of these elements in a manner similar to the upper portion of that cord 9′, 10′. Thus, the cords 9′ and 10′ loosely secure the rear panels 33′ and 35′ in place adjacent to the lateral sides 78′ and 79′ of the lumbar panel 3′.

Although FIGS. 6-6b depict a particular arrangement of features, it should be understood that alternative arrangements of the cords 9′ and 10′, the eyelets 81′ and 83′ and the anchor points 85′ and 87′ are also within the scope of the disclosed subject matter.

The inclusion of rollers 89′ in eyelets 81′ and 83′ may minimize friction in the cinching system 8′ and increase the amount of force that may be achieved with the cinching system 8′. The arrangement of the rollers 89′ spreads that force out over the height of the cinching system (e.g., the distance between an uppermost roller 89′ on one side of the lumbar support 3′ and a lowermost roller 89′ on that side of the lumbar support 3′, etc.). By increasing the force that may be applied with the cinching system 8′, the amount of leverage that may be achieved with the cord(s) 9′, 10′ of a cinching system 8′ is also increased. This added leverage enables the back orthosis 1 to be used in a manner that causes the lumbar panel 3′ to apply an increased and optimal amount of localized or focused and/or directional force to a wearer's back (e.g., as much as fifty percent (50%) to sixty percent (60%) more force than may be applied when the cinching system depicted by and described in reference to FIG. 5 is used). In addition, the configuration and/or dimensions of each receptacle 81R′ may prevent a cord 9′, 10′ that extends at least partially around the roller 89′ from sliding off of the roller 89′.

As shown, each roller 89′ may be configured as a pulley or spool. The divergent outward tapering from the center of a spindle to the rims at its ends facilitates retention of a cord 9′, 10′ that extends through each eyelet 81′, 83′ by the roller 89′ of that eyelet 81′, 83′. While the rollers 89′ shown in the drawings are shaped like pulleys or spools, other embodiments of rollers 89′ are also within the scope of this disclosure.

In the depicted embodiment, each roller 89′ and its corresponding pin 90′ may be positioned substantially parallel to its corresponding side 78′, 79′ of the lumbar panel 3′. Such an orientation will position the rollers 89′ and their corresponding pins 90′ substantially vertically when an individual wears the back orthosis 1, and substantially parallel to a pressure front applied by the lumbar panel 3′ to a wearer's back when the back orthosis 1 is used. Of course, other orientations of the rollers 89′ are also within the scope of the disclosed subject matter, including embodiments where rollers 89′ are oriented at angles that tailor the leverage at one or more locations between a rear panel 33′, 35′ and its corresponding side 78′, 79′ of the lumbar panel 3′, as well as orientations that minimize friction in adjusting the tension in the cords 9′ and 10′ and, thus, adjusting the relative locations of the rear panels 33′ and 35′ and the lumbar panel 3, as well as the amount of force applied by the lumbar panel 3′, the area to which force is applied by the lumbar panel 3′ and the direction in which the lumbar panel 3′ applies force to the back of a wearer. By orienting each roller 89′ vertically or in another suitable non-horizontal position, the cinching system 8′ will continue to function in the desired manner when the relative lateral position of one or both sets of rollers 89′ (i.e., those in eyelets 81′ and those in eyelets 83′) shifts (e.g., when tension is applied to either cord 9′, 10′, etc.). If rollers 89′ were oriented horizontally, any movement of one or more rollers 89′ out of plane from other rollers 89′ would detrimentally affect the manner in which the cinching system 8′ operates.

Referring to FIGS. 7a-c, each cinching system may allow for side-to-side movement and adjustment of lumbar panel 3 about the wearer's spinal region while wearing back orthosis 1 (FIG. 1) and without having to remove back orthosis 1, as shown by directional arrows H and J. FIG. 7a, for instance, shows a left-ward disposed position of lumbar panel 3. FIG. 7b shows a centrally disposed position of lumbar panel 3. FIG. 7c shows a right-ward disposed position of panel 3. However, any intermediate position along this continuum is possible in accordance with a given condition at a particular moment in time. For example, in many cases acute back pain sufferers experience a list in the spine to compensate for pain from an injury. The ability laterally to re-position lumbar panel 3, even at a moment's notice, allows back orthosis 1 to provide support over the related para-spinal soft tissue that requires stabilization, thereby reducing the force and severity of such list.

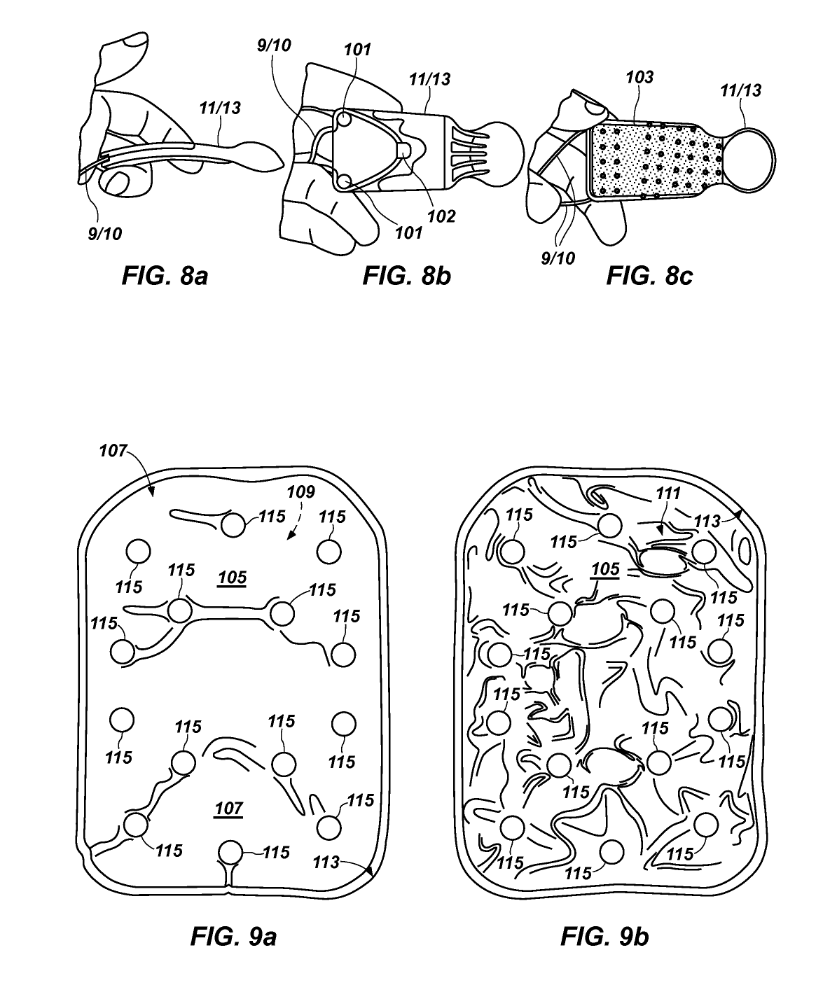

Referring to FIGS. 8a-c, each tensioning handle 11, 13, which may be curved, may create an offsetting tension in its respective cord 9, 10, when drawn. In particular, cord 9, 10 passes freely through its respective tensioning handle 11, 13 about an internal connector 101. Each tensioning handle 11, 13 has its own cord 9, 10 used to cinch each side of back orthosis 1 (FIG. 1). Having cord 9, 10 pass freely through tensioning handle 11, 13 may provide several advantages. First, it allows a wearer to self-adjust each cord 9, 10 between lumbar panel 3 and each rear panel 33, 35 (FIG. 2) to adapt to different waist and hip ratios and sizes from wearer to wearer. It also enables self-adjustment of cord 9, 10 to accommodate additional, or reduced, cord length (slack) when lumbar panel 3 is drawn to one lateral side versus the other so that the tensioning by tensioning handle 11, 13 is always pulling on both sides of its corresponding cord 9, 10 with equal force.

Free passage of each cord 9, 10 also in turn provides meaningful and repeatable vertical and circumferential attachment positioning along the entire area of attachment members 5 and 7. Thus, equally distributed tension makes a lower vertical attachment position correspond to a longer upper length of cord 9, 10, which tends to close the upper portion of the back orthosis 1 (FIG. 1) more, and vice versa.

Plus, this equally distributed, and greater more comfortable leverage, does not require handling the cords themselves. Limiting handling to tensioning handles 11 and 13 thus improves cinching ability by enhancing comfort and leverage—for all users. Curved handles also contour about a torso so as to lay flat when attached to attachment members 5 or 7 (FIG. 1) by hook fabric 103.

In one embodiment, both ends of each cord 9, 10 retract within its corresponding tensioning handle 11, 13 by the installation of suitable twin spring-loaded cord retraction devices. Such an installation, though independently taking-in cord slack on both sides of a handle, will not necessarily offer all of the advantages of the above-mentioned device, however.

Referring to FIGS. 9a and 9b, hot or cold gel pads (not shown), or foam pad 105 containing flowing foam, may be attached to the inner side of panel 3 (FIG. 1), for example, by hook-friendly fabric 107, which covers most of one side of pad 105. Side panels 109 and 111, which are made from a polyurethane film laminated to a brushed nylon fabric, are thermally bonded along edge 113 and circular press-points 115. As seen in FIG. 9b, pad 105 contains a paste-like foam, made by Tekpad (as described, e.g., in U.S. Pat. No. 6,583,199 herein incorporated by this reference in its entirety), that fills in the lower profile areas of a wearer's lumbar spine when worn in conjunction with back orthosis 1 (FIG. 1). However, any material suitable for filling a void into which it flows may be used, including other fluids and gases of various densities.

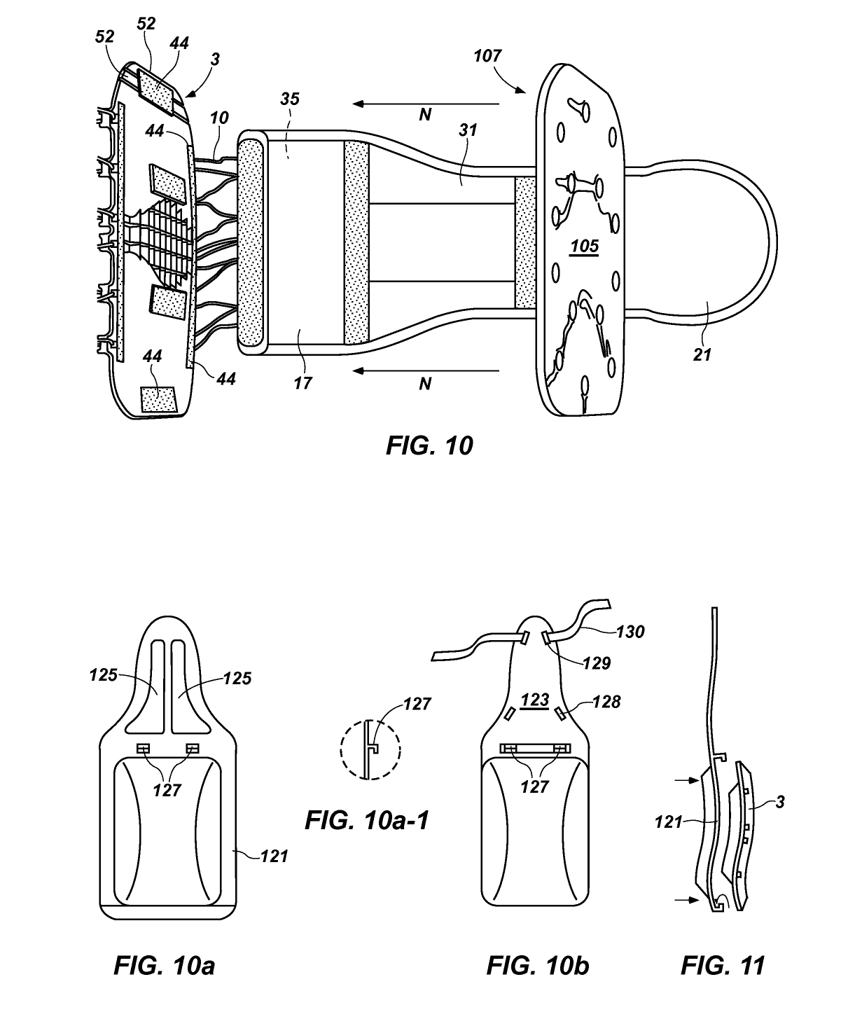

Referring to FIG. 10, pad 105 attaches to panel 3 as shown in accordance with direction arrows N.

Referring to FIG. 10a, thoracic lumbo sacral orthosis (“TLSO”) attachment plate 121 having apertures 125, and made from injection molded nylon, fits on the same inner side of panel 3 to form a thoracic lumbo sacral orthosis. Attachment plate 121 is a prefabricated piece that accepts lumbar panel 3 by a snap fit. Plastic clip tabs 127 reversibly snap into slots 52 (see FIGS. 1 and 2) on lumbar panel 3.

Referring to FIG. 10b, in an alternate embodiment, TLSO attachment plate 123 comprises a solid upper plate without apertures. Buckle or other attachment point 128 accepts strap 130 after strap 130 is attached from slots 129 and wrapped around a wearer's, e.g., shoulders and armpits.

Referring to FIG. 11, in either embodiment lip 131 fits over the bottom edge of lumbar panel 3 so that clip tabs 127 may snap into slots 52. Lumbar panel 3 and attachment plate 123 fit together so that they both contour to the curvature of a spine during their application. This drawing, for example, shows a curve when the wearer's lumbar region is convex, such as when bending over.

In one embodiment, an orthoarthritic, thoracic fractures and thoracic surgeries attachment panel is similarly attached, in particular to afford posterior application of proximal ends of straps that run over a wearer's shoulders and/or around the armpit area for attachment at their distal ends upon the orthoarthritic, thoracic fractures and thoracic surgeries panel.

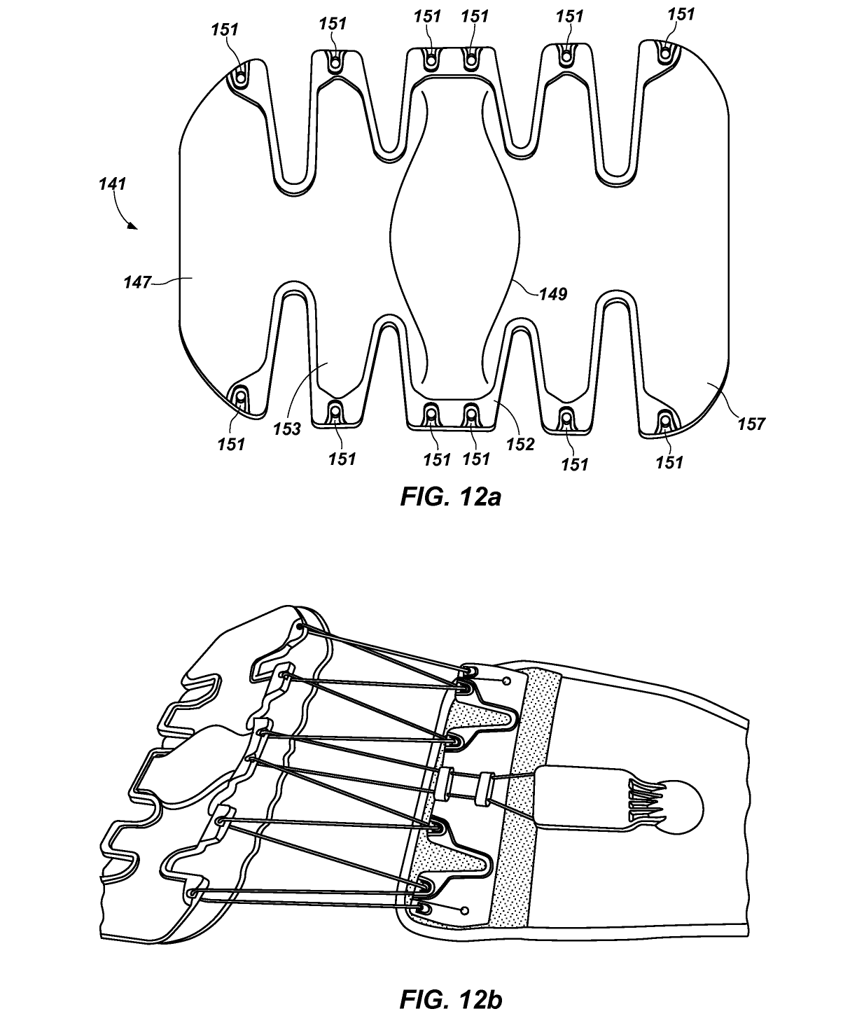

Referring to FIGS. 12a and 12b, in one embodiment lumbar panel 141 is used in place of lumbar panel 3. Lumbar panel 141 generally has the same outside perimeter shape and size, and material composition, hardness and thickness, as lumbar panel 3, but instead of two bowls its inner side is generally flat. It has four slots on each side of the panel to enable a 147 of lumbar panel 141, which runs down the entire central vertical length of lumbar panel 141, to flex toward the spine so as to fit into the lordatic curve of the lumbar spine when sufficient tension is applied to the cords. In addition to this vertical flexing of central vertical column 147, ribs 153 and 157 of lumbar panel 141 may independently flex transversely and/or horizontally, either at the same time or at different times than the vertical flexion of column 147. Each rib 153 and 157 therefore has a slight lip 152 (e.g., a 0.0020 inch lip) along its edge so as not to fatigue and split with repetitive flexions, as well as a raised support center 149 that provides added stability and strength.

A second aspect of the disclosure includes an orthotic method that includes any of the components and techniques substantially as described above. Other embodiments, techniques or devices can also or alternately be used in this method aspect.

While it is apparent that the illustrative embodiments of the subject matter disclosed herein fulfill the limitations of the appended claims, it is appreciated that numerous modifications and other embodiments may be devised by those skilled in the art. Additionally, feature(s) and/or element(s) from any embodiment may be used singly or in combination with other embodiment(s). Therefore, it will be understood that the appended claims are intended to cover all such modifications and embodiments that would come within the broader spirit and scope of the disclosed concepts.

1. A back orthosis, comprising:
a pair of rear anchors, each rear anchor of the pair being configured to be oriented substantially vertically and positioned over and secured against a lateral location on a back of a torso of a subject, the pair of independent rear anchors being configured to be positioned over and secured against lateral locations on opposite sides of and laterally spaced apart from a spine of the back;
a lumbar support positioned between the pair of independent rear anchors and oriented to be positioned over and secured against the spine to apply focused pressure along the spine while applying substantially no pressure to elongated locations of the back oriented substantially parallel to and located laterally adjacent to opposite sides of the spine; and
a pair of independent cinching systems, each cinching system of the pair:
being associated with a rear anchor and an adjacent lateral portion of the lumbar support located adjacent to that rear anchor; and
configured to define a distance between adjacent edges of the rear anchor and the adjacent lateral portion of the lumbar support,
the pair of independent cinching systems configured to operate together to define an amount of the focused pressure applied along the spine.

2. The back orthosis of claim 1, wherein portions of each cinching system of the pair that extend across the elongated locations of the back extend across the elongated locations while applying substantially no pressure to the elongated locations.

3. The back orthosis of claim 1, comprising:
an anterior element configured to be positioned over at least a portion of an anterior of the torso of the subject.

4. The back orthosis of claim 3, wherein the anterior element is configured to exert force against the anterior surface of the torso toward the spine.

5. The back orthosis of claim 4, wherein the anterior element and the pair or rear anchors are configured and arranged to apply force to the torso at locations that decompress the spine.

6. The back orthosis of claim 3, wherein the pair of rear anchors are located at or adjacent to opposite ends of the anterior element.

7. The back orthosis of claim 1, wherein each cinching system includes:
a first plurality of rollers at or adjacent to a medial edge of the rear anchor;
a second plurality of rollers at or adjacent to a lateral edge of the lumbar support that opposes the medial edge of the rear anchor; and
at least one cord:
carried by each roller of the first plurality of rollers and the second plurality of rollers;
extending between the first plurality of rollers and the second plurality of rollers; and
adjustably assembling the rear anchor and the lumbar support.

8. The back orthosis of claim 7, wherein each roller of the first plurality and the second plurality is configured to be oriented non-horizontally when the lumbar support and the rear anchor are positioned on a torso of a subject.

9. The back orthosis of claim 8, wherein each roller is configured to be oriented substantially vertically when the lumbar support and the rear anchor are positioned on a torso of a subject.

10. The back orthosis of claim 1, wherein the rear anchors are configured to be oriented at an angle to one another.

11. The back orthosis of claim 1, wherein an angle at which the rear anchors are oriented to one another and an amount of pulling force applied by each cinching system to its corresponding lateral edge of the lumbar support at least partially define the amount of the focused pressure applied to the spine.

12. A back orthosis, comprising:
at least one support configured to be positioned at least over at least a portion of a front side of a torso of a subject;
a lumbar support configured to be positioned over a portion of a back of the torso;
a pair of cinching systems, each cinching system:
extending between an outer edge of the at least one support and a lateral edge of the lumbar support; and
comprising:
a first plurality of rollers at or adjacent to the outer edge of the at least one support;
a second plurality of rollers at or adjacent to a lateral edge of the lumbar support that opposes the outer edge of the at least one support; and
at least one cord:
carried by each roller of the first plurality of rollers and the second plurality of rollers;
extending between the first plurality of rollers and the second plurality of rollers; and
adjustably assembling the rear anchor and the lumbar support.

13. The back orthosis of claim 12, wherein the at least one support is further configured to extend over lateral portions of a torso of a subject and comprises a pair of rear anchors configured to be positioned over lateral portions of the back of a torso.

14. The back orthosis of claim 12, wherein each roller of the first plurality and the second plurality is configured to be oriented non-horizontally when the lumbar support and the at least one support are positioned on a torso of a subject.

15. The back orthosis of claim 14, wherein each roller is configured to be oriented substantially vertically when the lumbar support and the at least one support are positioned on a torso of a subject.

16. The back orthosis of claim 12, wherein the pair of cinching systems are configured to be oriented at an angle to one another.

17. The back orthosis of claim 16, wherein the angle at which the pair of cinching systems are oriented to one another and an amount of pulling force applied by each cinching system to its corresponding lateral edge of the lumbar support at least partially define an amount of the focused pressure applied by the lumbar support to the spine.

18. A method for selectively applying focused pressure to a spine, comprising:
positioning a lumbar support on a back of a torso of a subject;
placing a support across at least a portion of a front side of the torso;
introducing tension into each cinching system of a pair of cinching systems that assemble outer edges of the support to lateral edges of the lumbar support,
the tension in each cinching system causing that cinching system to exert a pulling force on its corresponding lateral edge of the lumbar support,
angles at which the cinching systems are oriented relative to one another or relative to the lumbar support and an amount of the pulling force applied by each cinching system to its corresponding lateral edge of the lumbar support at least partially defining an amount of focused pressure applied by the lumbar support to the spine.

19. The method of claim 18, wherein introducing tension is effected by introducing tension into a system in which at least one cord is secured between the support and the lumbar support by a plurality of non-horizontally oriented rollers positioned adjacent to each outer edge of the support and each lateral edge of the lumbar support and along which the at least one cord travels.

20. The method of claim 18, wherein introducing tension includes introducing tension between rear anchors secured in place on lateral portions of a back of a torso of a subject and the lateral edges of the lumbar support.
